(19) United States
(12) Patent Application Publication
(10) Pub. No.: US 2026/0007846 A1
(43) Pub. Date: Jan. 8, 2026

(54) ENDOTRACHEAL TUBE

(71) Applicants: Joshua Joseph Herskovic, BOCA RATON, FL (US); HANNAH F HERSKOVIC, BOCA RATON, FL (US)

(72) Inventors: Joshua Joseph Herskovic, BOCA RATON, FL (US); HANNAH F HERSKOVIC, BOCA RATON, FL (US)

(21) Appl. No.: 19/257,253

(22) Filed: Jul. 1, 2025

Related U.S. Application Data
(60) Provisional application No. 63/667,103, filed on Jul. 2, 2024.

(51) Int. Cl. A61M 16/04; A61B 1/00; A61B 1/04; A61B 1/267; A61M 16/00

(52) U.S. Cl. CPC A61M 16/0418; A61B 1/00009; A61B 1/00016; A61B 1/0004; A61B 1/00052; A61B 1/04; A61B 1/267; A61M 16/024; A61M 2205/33; A61M 2205/3592; A61M 2205/502; A61M 2210/065

(57) ABSTRACT

Disclosed is an endotracheal tube system having an endotracheal tube and a control system to provide navigational elongated endotracheal tube to facilitate bending into trachea. The elongated endotracheal tube includes a hollow inner diameter wall, an outer diameter wall, plurality of slots, and a bending section. The plurality of slots configured to be form across length of the hollow inner diameter wall and the outer diameter wall. The bending section is configured to reduce amount of force required to bend the hollow inner diameter wall and the outer diameter wall. The control system includes a base plate, plurality of attachment units, plurality of navigational wires runs through at least one slot across length of the elongated endotracheal tube and loops back via the slots at the bending section. The base plate is configured to pull at least one of the plurality of navigational wires to navigate the endotracheal tube.

### CROSS REFERENCE TO RELATED APPLICATION

This application claims priority to U.S. Provisional Patent App. Ser. No. 63/667,103 filed on Jul. 2, 2024 entitled “Remote Steerable Intubation Tube” which is hereby incorporated by reference in their entirety as though fully and completely set forth herein.

### BACKGROUND OF THE INVENTION

### 1. Field of the Invention

The present invention generally relates to an endotracheal tube (ETT), and more particularly relates to a navigational endotracheal tube navigated into the trachea via a joystick.

### 2. Description of Related Art

Tracheal intubation is a medical technique in which a breathing tube is carefully inserted through the mouth, past the vocal cords and larynx, and down into the windpipe. To assist with visualizing the airway opening during this process, healthcare providers typically use a laryngoscope. The tube itself usually features an inflatable cuff near its tip, which, once expanded, creates a secure seal against the inner lining of the trachea. This seal helps to block fluids from entering the lungs, thereby reducing the risk of aspiration.

A traditional stylet, as known in the prior art, comprises a manually malleable piece of metal that is configured to be inserted into the lumen of an endotracheal tube (ETT). The stylet imparts a desired curvature to the endotracheal tube, allowing the tube to maintain a predetermined bend contour. This curvature facilitates placement of the endotracheal tube by conforming the tube to the anatomical pathway of the patient's airway, thereby assisting in intubation procedures.

To help a medical professional view a patient's airway anatomy, tools such as a laryngoscope, video laryngoscope, or similar devices are commonly employed. Once the intubation procedure is completed, the endotracheal tube remains in place to maintain an open airway for airflow through the respiratory tract. Conventional stylets, often part of prior art, consist of manually bendable metal rods that are inserted into the endotracheal tube. These stylets allow the tube to retain a specific curved shape, which facilitates the insertion of the tube by aligning its form with the natural curvature of the patient's airway.

In patients with challenging airway anatomy, endotracheal intubation is often facilitated using a video laryngoscope. The scope is advanced into the hypopharynx, until the glottis opening is visualized. Because a view of the glottis opening is more easily obtained using video laryngoscopy than a conventional direct laryngoscopy this method is typically preferred by users with lower intubation proficiency.

Despite the improvement of visualization of the glottic opening a secondary problem is created in which it is now more difficult to pass the endotracheal tube into the glottic opening when compared to direct laryngoscopy. Several solutions have been attempted with the most commonly used solution to be a rigid stylet inserted into the ETT that may maintain a more acute bend that approximates the curvature of the hypopharynx. Because of its rigidity it maintains its bend angle while transversing the soft tissue, this results in making it easier to bring the ETT in close approximation to the glottic opening.

However, the operator has difficulty getting the ETT to pass through the glottic opening and into the trachea. Typically, the ETT will get trapped just behind the glottic opening on a structure termed the arytenoids. A second obstacle is encountered one the operator is able to proceed past the arytenoids. The rigid stylet does not allow the user to directly guide the endotracheal down and into the trachea.

Furthermore, once the endotracheal tube is inserted into the airway along with the stylet, the stylet can pose a risk of trauma to the vocal cords or trachea. Therefore, there is a need of an endotracheal tube system providing having a navigational bending section. Further, the endotracheal tube system should include a control system to selectively bend the bending section in multiple directions to reach the trachea.

### SUMMARY OF THE INVENTION

In accordance with an embodiment of the present invention, an endotracheal tube system is provided.

An object of the present invention is to provide an endotracheal tube system with an elongated endotracheal tube and a control system. The elongated endotracheal tube is having a hollow inner diameter wall, an outer diameter wall, plurality of slots, and a bending section. The control system is configured to have a base plate, plurality of attachment units, and plurality of navigational wires.

The outer diameter wall is equidistantly surrounding the hollow inner diameter wall. The plurality of slots is configured uniformly between the outer diameter wall and the hollow inner diameter wall. The plurality of slots is configured to form across length of the hollow inner diameter wall and the outer diameter wall.

The bending section is configured near the open distal end of the elongated endotracheal tube. The bending section is configured to reduce amount of force required to bend the hollow inner diameter wall and the outer diameter wall. The control system is configured to attach with the elongated endotracheal tube.

The base plate is configured to detachably attach to the elongated endotracheal tube. The base plate is having a top surface, a bottom surface, a tracheal tube airway connector configured on the top surface, and an endotracheal tube receptacle configured on the bottom surface to receive the elongated endotracheal tube. The plurality of attachment units are configured to attach the base plate with the endotracheal tube.

The plurality of navigational wires includes a first navigational wire and a second navigational wire. The first navigational wire is having an elongated first wire body having a first wire top end securely attaches to a first attachment unit and a first wire distal end. The elongated first wire body runs through at least one slot across length of the elongated endotracheal tube and loops back via the slots at the bending section to securely attach with a second attachment unit at the first wire distal end.

The second navigational wire having an elongated second wire body having a second wire top end securely attaches to a third attachment unit and a second wire distal end. The elongated second wire body runs through at least one slot across length of the elongated endotracheal tube and loops back via the slots at the bending section to securely attach with a fourth attachment unit at the second wire distal end. The base plate is configured to pull at least one of the plurality of navigational wires to navigate the distal end of the endotracheal tube.

Another object of the present invention is to provide the control system with wire anchors to attach top end of the navigational wires to the base plate.

Another object of the present invention is to provide the control system with an electrical coupling unit configured to attach with the base plate to conduct power to the navigational wires.

Another object of the present invention is to provide the endotracheal tube system with a navigational control unit for navigating the elongated endotracheal tube.

Another object of the present invention is to provide the navigational control unit with a navigational input unit to provide navigational signals for the navigational wires, a microcontroller to process the navigational signals, plurality of electrical switches to direct power to the navigational wires under the direction of the microcontroller, and a power source.

Another object of the present invention is to provide navigational input unit with a laryngoscope housing, a display unit, a microcontroller, an electric button and a communication unit.

Another object of the present invention is to provide the endotracheal tube system wherein the first navigational wire shortens in length on application of directed power based on the navigational input unit, and the second navigational wire moves via the slots to center of curvature of the elongated endotracheal tube to prevent resistance to bending.

Another object of the present invention is to provide the endotracheal tube system with anchor points to facilitate bending of the bending section into the trachea in four different directions.

### BRIEF DESCRIPTION OF DRAWINGS

The annexed drawings, which are not necessarily to scale, show various aspects of the inventions in which similar reference numerals are used to indicate the same or similar parts in the various views.

FIG. 1A illustrates an exploded view of an endotracheal tube system;

FIG. 1B illustrates a bottom view of the endotracheal tube system;

FIG. 1C illustrates an exploded view of the endotracheal tube system in another embodiment of the present invention;

FIG. 2A illustrates a perspective view of the elongated endotracheal tube;

FIG. 2B illustrates a cross-section view of the elongated endotracheal tube;

FIG. 2C illustrates a side perspective view of the bending section of the elongated endotracheal tube;

FIG. 2D illustrates an exploded view of the elongated endotracheal tube in another embodiment of the present invention;

FIG. 3A is a schematic view to showcase position of the bending section, the electric button, position of navigational wires in the slot;

FIG. 3B is another schematic view to showcase position of the bending section, the electric button, position of navigational wires in the slot in another embodiment of the present invention;

FIG. 3C is another schematic view to showcase position of the bending section, the electric button, position of navigational wires in the slot in another embodiment of the present invention;

FIG. 3D is another schematic view to showcase position of the bending section, the electric button, position of navigational wires in the slot in another embodiment of the present invention;

FIG. 4A illustrates a schematic view of movement of the base plate 106 on the elongated endotracheal tube;

FIG. 4B illustrates a schematic view of movement of the base plate 106 on the elongated endotracheal tube in another embodiment of the present invention;

FIG. 5A illustrates a schematic view of an elongated endotracheal tube in another embodiment of the present invention; and

FIG. 5B illustrates a schematic view of the elongated endotracheal tube showing anchor points.

### DETAILED DESCRIPTION OF DRAWINGS

Reference will now be made in detail to an implementation consistent with the present invention as illustrated in the accompanying drawings. Wherever possible, the same reference numbers will be used throughout the drawings and the following description to refer to the same or like parts. Although discussed with reference to these illustrations, the present invention is not limited to the implementations illustrated therein. Hence, the reader should regard these illustrations merely as examples of embodiments of the present invention, the full scope of which is measured only in terms of the claims following this description.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

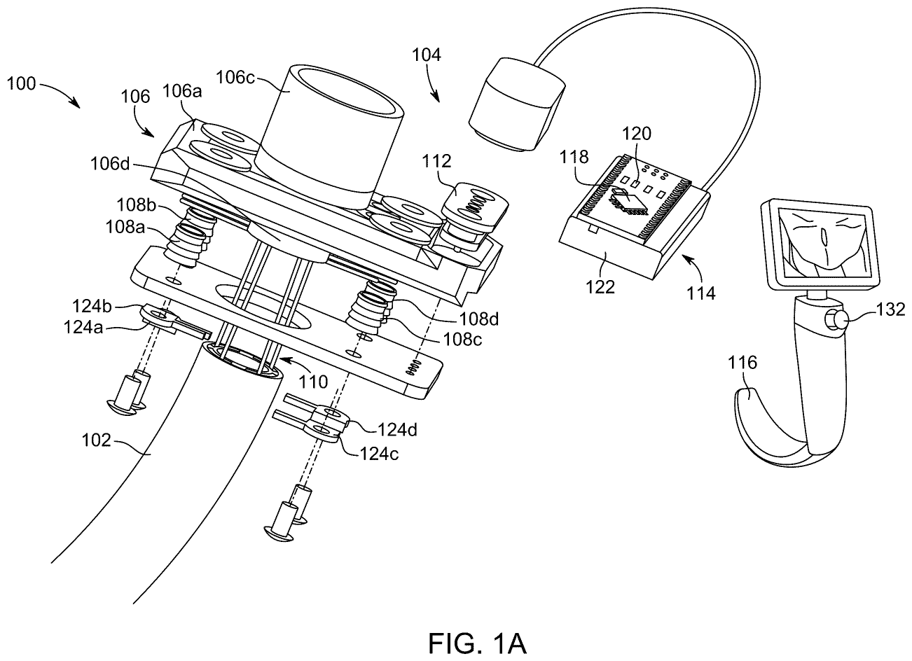

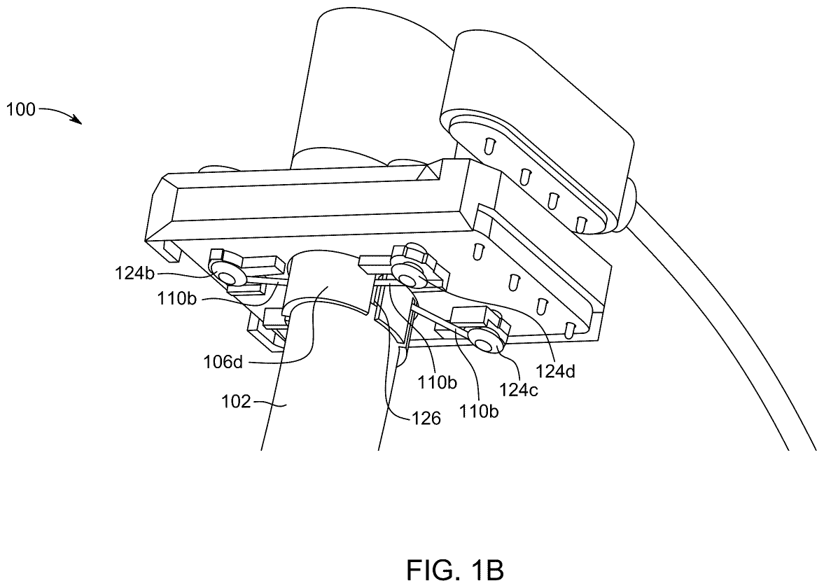

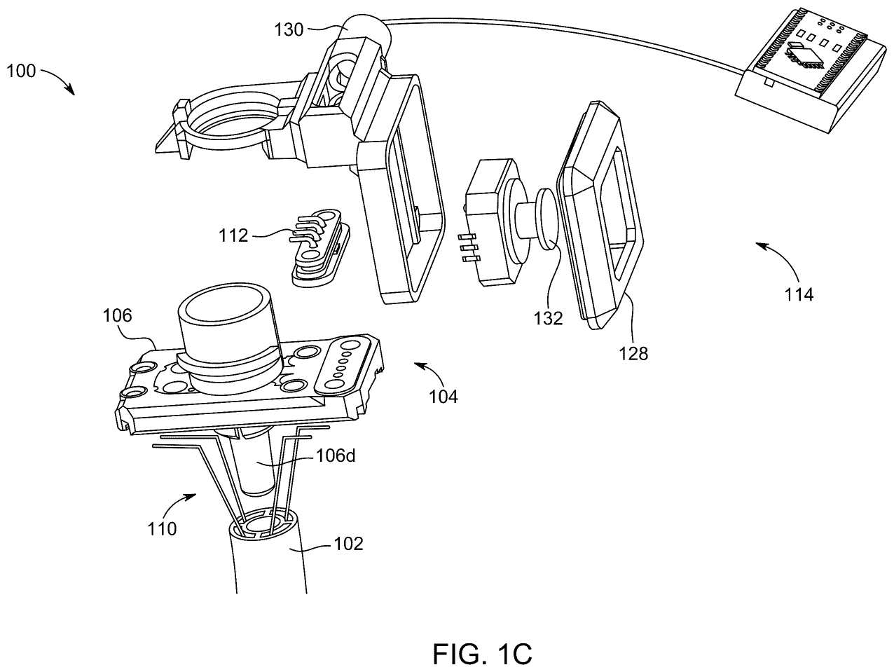

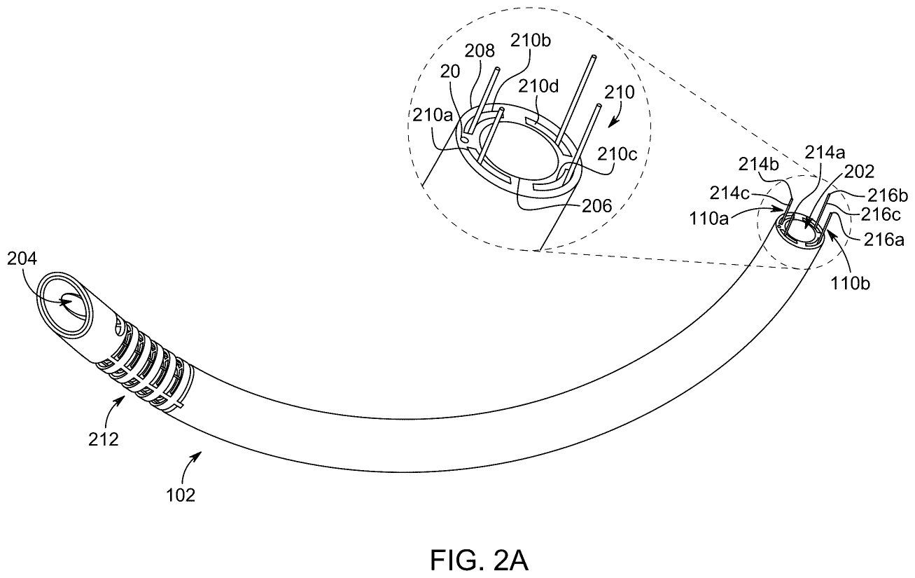

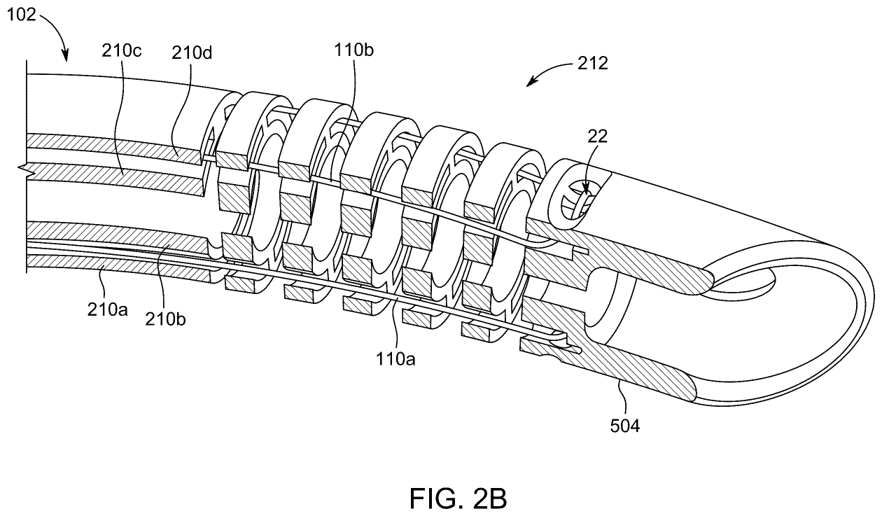

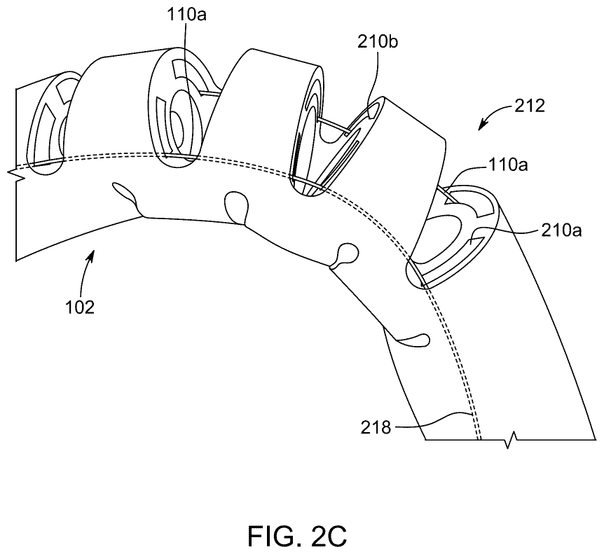

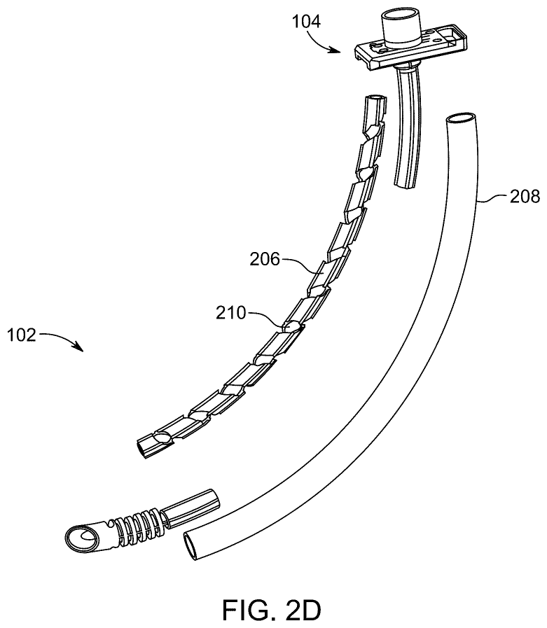

FIG. 1A illustrates an exploded view of an endotracheal tube system 100. The endotracheal tube system 100 includes an elongated endotracheal tube 102 and a control system 104 is configured to attach with the elongated endotracheal tube 102. The elongated endotracheal tube 102 is explained in detail in conjunction with FIG. 2 of the present invention.

The control system 104 includes a base plate 106, plurality of attachment units 108 such as 108a, 108b, 108c, and 108d, and plurality of navigational wires 110. The base plate 106 is configured to attach to the elongated endotracheal tube 102. The base plate 106 is having a top surface 106a, a bottom surface (106b, shown in FIG. 1B), a tracheal tube airway connector 106c is configured on the top surface 106a, and an endotracheal tube receptacle 106d is configured on the bottom surface to receive the elongated endotracheal tube 102.

The tracheal tube airway connector 106c is configured detachably connect the endotracheal tube 102 to a breathing circuit or other equipment like a ventilator or resuscitator. The endotracheal tube receptacle 106d is explained in detail in conjunction with FIG. 1B of the present invention.

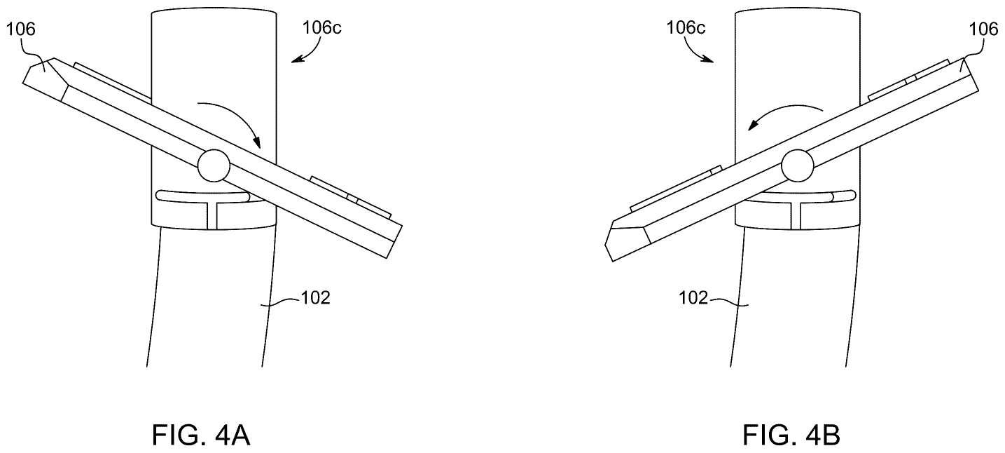

The base plate 106 is configured to pull at least one of the plurality of navigational wires 110 to navigate the distal end of the endotracheal tube 102. In an embodiment of the present invention, the base plate 106 is mechanically controlled by a user. The mechanically controlled base plate 106 is shown in FIGS. 4A and 4B of the present invention.

In another embodiment, the control system 104 further includes an electrical coupling unit 112 configured to attach with the base plate 106 to conduct power to the navigational wires 110. Examples of the electrical coupling unit 112 include but not limited to a magnetic coupling, pogo connectors, electrical pins, etc. It will be apparent to those skilled in the art that various types of electrical coupling unit 112 may be envisioned to direct power to navigational wires 110 without deviating from the scope of the present invention.

In another embodiment of the present invention, the endotracheal tube system 100 further includes a navigational control unit 114 configured to attach with the control system 104 to navigate the elongated endotracheal tube 102. The navigational control unit 114 further includes a navigational input unit 116, a microcontroller 118, plurality of electrical switches 120, and a power source 122.

The navigational input unit 116 provides navigational signals to operate the navigational wires 110. The navigational signals may be generated either wirelessly or wired. Examples of the navigational input unit 116 include but not limited to a video laryngoscope, electric/navigator buttons, remote controlled electric buttons, wired electric buttons, microprocessor analysis or programmed based electric buttons, etc.

In one embodiment, the navigational input unit 116 is a video laryngoscope. The video laryngoscope includes a display unit (screen) to display video signals from a camera, an electric button 132 to provide navigation instructions to the microcontroller 118. The microcontroller 118 then sends the navigation instructions to the navigational wires 110 through the electrical switches 120. Examples of the video signals include but not limited to videos, images, processed video data from the microprocessor for navigation of the endotracheal tube, etc. The video laryngoscope further includes a communication unit to communicate navigational signals over a communication network. Examples of the communication unit includes but not limited to a Bluetooth, Wi-Fi, and other form of radio communication, LAN, and IR communication.

The microcontroller 118 is operably coupled to the navigational input unit 116 to process the navigational signals. Examples of the microcontroller 118 include but not limited to an 8051 microcontroller, a Peripheral Interface controller (PIC), an Atmel AVR microcontroller, an Advanced Rise Machines (ARM) processor, a reduced instruction set computing (RISC) processor, a complex instruction set computing (CISC) processor, and the like. It will be apparent to a person skilled in the art that various types of the microcontroller 118 compatible with multiple operating systems may be envisioned without deviating from the scope of the present invention.

The plurality of electric switches 120 is configured to direct the power to the navigational wires 110 through the electrical coupling unit 112. Further, the plurality of electrical switches 120 directs or conducts power based on the processed navigational signals. In another embodiment, the plurality of electrical switches 120 are controlled by the microcontroller 118. The microcontroller 118 provides variable frequency switching instructions to the electrical switches 120.

Examples of the electrical switches 120 include but not limited to a transistor, a diode (e.g., a PN diode, a PIN diode, a Schottky diode, a metal-insulator-metal (MIM) diode, a metal-insulator-semiconductor (MIS) diode, or a diode-connected transistor), metal-oxide-semiconductor field-effect transistor (MOSFET), and a logic circuit in which such elements are combined.

The power source 122 is configured to provide power to the microcontroller 118, the navigational input unit 116, and the plurality of electrical switches 120. Examples of the power source 122 include but not limited to a radio frequency (RF) power supply, a battery, a battery pack, a programmable power supply, alternating power, electromagnetic induction and electromagnetic field resonance.

It will be apparent to those skilled in the art that various types of the navigational input unit 116, the microcontroller 118, the electric switches 120, and the power source 122 may be envisioned without deviating from the scope of the present invention.

In another embodiment, the control system 104 further includes wire anchors 112 such as wire anchor 124a, 124b, 124c and 124d to attach proximal or top end of the navigational wire 110 to the base plate 106. The wire anchors 112 are further explained in detail in conjunction with FIG. 1B of the present invention.

FIG. 1B illustrates a bottom view of the endotracheal tube system 100. The navigational wires 110 are attached to the base plate 106 through the attachment units 108. In an embodiment, the plurality of navigational wires includes a first navigational wire 110a, and a second navigational wire 110b.

The second navigational wires 110b is attached to the wire anchor 124c at a second wire top end (shown in FIG. 2A) and wire anchor 124d at a second wire distal end (shown in FIG. 2A), respectively. Further, the navigational wire 110a is attached to the wires anchor (124a shown in FIG. 1A) at a first wire top end (shown in FIG. 2A) and the wire anchor 124b at a first wire distal end (shown in FIG. 2A), respectively.

In an embodiment, the endotracheal tube receptacle 106d is configured to have recesses 126 to allow passage for the navigational wires 110 to attach with the wire anchors 124. It would be readily apparent to those skilled in the art that the endotracheal tube receptacle 106d may be envisioned in various forms, shapes & sizes without deviating from the scope of the present invention.

FIG. 1C illustrates an exploded view of the endotracheal tube system 100 in another embodiment of the present invention. In an embodiment, the navigational input unit 114 is electrically connected to the base plate 106. The navigational input unit 114 includes a top frame 128, a bottom frame 130 attached to the base plate 106 via the electrical coupling unit 112, and an electric button 132 allows a user to selectively power one of the plurality of navigational wires 110.

The electric button 132 is configured to position between the top frame 128 and the bottom frame 130. The electric button 132 sends navigational signals to the navigational wires 110. Examples of the electric button 132 but not limited to a toggle switch, a joystick, a rotatory switch, a tactile switch, a push switch, a slider switch, a float switch, a flow switch, etc.

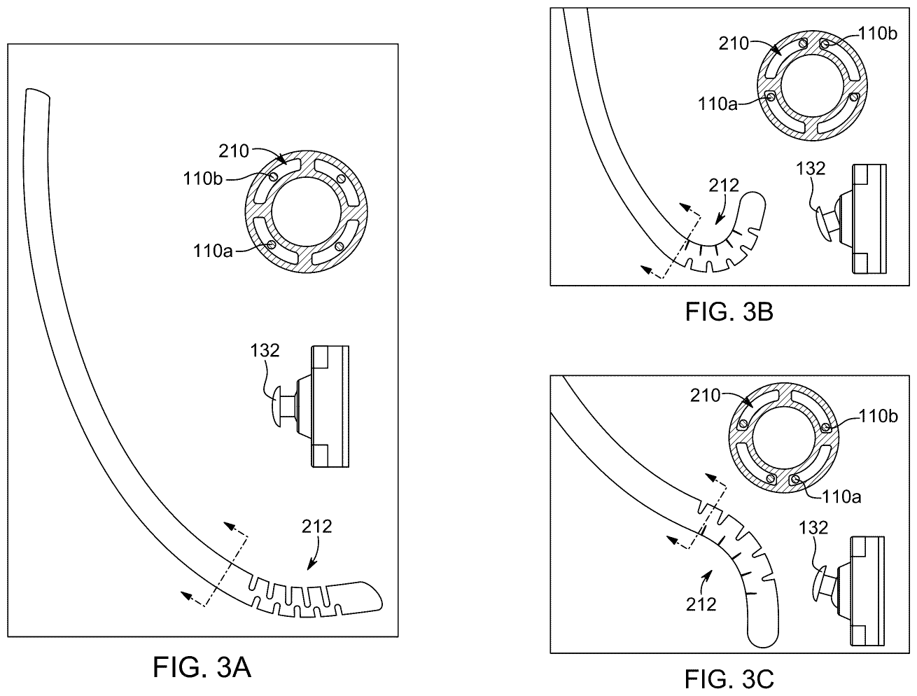

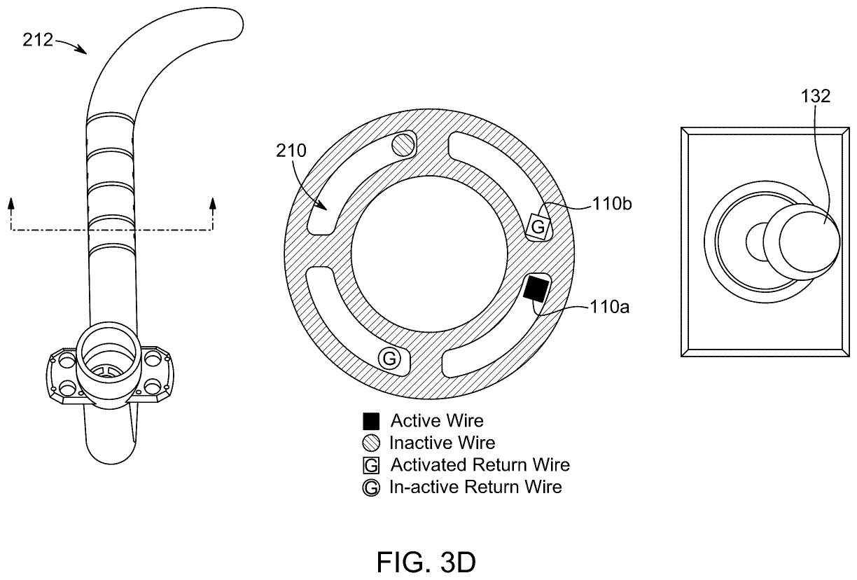

It will be readily apparent to those skilled in the art that various types of electric button 132 may be envisioned without deviating from the scope of the present invention. The navigational signals are explained in detail in conjunction with FIGS. 3A, 3B, 3C and 3D of the present invention.

FIG. 2A illustrates a perspective view of the elongated endotracheal tube 102. The elongated endotracheal tube 102 have an open top end 202, and an open distal end 204. The elongated endotracheal tube 102 includes a hollow inner diameter wall 206, an outer diameter wall 208, plurality of slots 210 and a bending section 212.

The outer diameter wall 208 is equidistantly surrounding the hollow inner diameter wall 206. The plurality of slots 210 is configured uniformly between the outer diameter wall 208 and the hollow inner diameter wall 206. The plurality of slots 210 is configured to be form across length of the hollow inner diameter wall 206 and the outer diameter wall 208.

The plurality of slots 210 such as 210a, 210b, 210c and 210d are uniform i.e. the slots 210 are of same shape and size. The formation of slots 210 across length of the hollow inner diameter wall 206 and the outer diameter wall 208 is shown in detail and explained through FIG. 2B of the present invention.

The bending section 212 is configured near the open distal end 204 of the elongated endotracheal tube 102. The bending section 212 is configured to reduce amount of force required to bend the hollow inner diameter wall 206 and the outer diameter wall 208.

The bending section 212 may be configured in many shapes such as helical shape, spiral shape, corrugated, and notches. The shape of the bending section 212 may be configured by creating notches that results in removing of certain hollow inner diameter wall 206 or the outer diameter wall 208 or both the inner wall and the outer wall within the bending section 212. It will be readily apparent to those skilled in the art that various shapes of bending section 212 may be envisioned without deviating from the scope of the present invention.

The plurality of navigational wires 110, wherein a first navigational wire 110a is having a first wire top end 214a, a first wire distal end 214b, an elongated first wire body 214c is configured between the first wire top end 214a and the first wire distal end 214b. The elongated first wire body 214c runs through at least one slot 210a across length of the elongated endotracheal tube 102 and loops back via the slot 210b at the bending section 212 to securely attach with the second attachment unit (108b, shown in FIG. 1A).

Similarly, a second navigational wire 110b is having a second wire top end 216a, a second wire distal end 216b, an elongated second wire body 216c is configured between the second wire top end 216a and the second wire distal end 216b. The elongated second wire body 216c runs through slot 210c across length of the elongated endotracheal tube 102 and loops back via the slot 210d at the bending section 212 to securely attach with the fourth attachment unit (108d, shown in FIG. 1A).

FIG. 2B illustrates a cross-section view of the elongated endotracheal tube 102. The endotracheal tube 102 has four slots 210a, 210b, 210c and 210d. The first navigational wire 110a runs through slot 210a across length of the elongated endotracheal tube 102 and loops back via the slot 210b at the bending section 212.

Similarly, the second navigational wire 110b runs through slot 210c across length of the elongated endotracheal tube 102 and loops back via the slot 210d at the bending section 212. The looping back 216 of the navigational wires 110 is visible clearly in the cross-section view.

FIG. 2C illustrates a side perspective view of the bending section 212 of the elongated endotracheal tube 102. The power is directed to shorten the length of the second navigational wire 110b (active wire) resulting in downward flexion and in order to prevent resistance to bending the first navigational wire 110a (non-active) moves to the center of curvature 218 (shown as axis) via the slot 210a and slot 210b.

FIG. 2D illustrates an exploded view of the elongated endotracheal tube 102 in another embodiment of the present invention. The hollow inner diameter wall 206 is configured with slots 210 throughout the length of the elongated endotracheal tube 102. The slots 210 are explained in detail in conjunction with FIG. 2A to 2C of the present invention.

The slots 210 are configured to create space or indents on the elongated endotracheal tube 102, which when assembled with the outer diameter wall 208 forms slots 210 for receiving the navigational wires. The control system 104 attaches to the elongated endotracheal tube 102. The control system 104 is explained in detail in conjunction with FIG. 1A to 1C to present invention.

Examples of the material of navigational wires 110 include but not limited to shape memory alloy (SMA), nitinol, and other similar contractile materials, or pull wires. A limiting property of SMA as an actuator is its ability to return to its initial length, its non-contracted state, is dependent on the time for it to cool past its transition temperature. The ability of SMA to cool by dissipating heat is directly proportional to its outer diameter.

The navigational wires 110 includes slack in the wire so that when a user bends the elongated endotracheal tube (ETT) manually it does not result in flexion of the bending section. If nitinol is being used as the actuator it's stroke length is limited to ˜4% of the length of SMA length. Thus, slack must be significantly less than 14 mm because a significant portion of this stroke is devoted to flexion of the ETT.

A further property of SMA navigational wire is its contractile property is dependent on reaching a transition temperature. Although the transition temperature is independent of the diameter of the SMA; the amount of heat required to reach the transition temperature is directly proportion to the diameter. Because SMA is a resistive element; heat is generated by passing an electrical current thru the SMA wire. The amount of current required to produce contraction is directly proportional to the diameter of the SMA wire.

FIGS. 3A, 3B, 3C and 3D are schematic views to showcase position of the bending section 212, the electric button 132, position of navigational wires 110 in the slot 210. As shown in FIG. 3A, the electric button 132 is in center position resulted in the navigational wires 110a, 110b at center of the slots 210, which maintains the position of the bending section 212.

As shown in FIG. 3B, the electric button 132 is down position which results in moving the first navigational wire 110a to be at top of the slots 210 and moving the second navigational wire 110b to be at top of the slots 210 which further results in upward movement of the bending section 212.

As shown in FIG. 3C, the electric button 132 is in up position which results in moving the first navigational wire 110a to be at bottom of the slots 210, and moving the second navigational wire 110b to be at bottom of the slots 210 which further results in downward movement of the bending section 212.

FIG. 3D showcase an embodiment with four navigational wires. The electric button 132 is in right position which results in moving the ‘active wire’ to the top of the slot, and the ‘activated return wire’ at bottom of the slot. Further, the ‘inactive wire’ is at the top of the slot and ‘in-active return wire’ is at bottom of the slot.

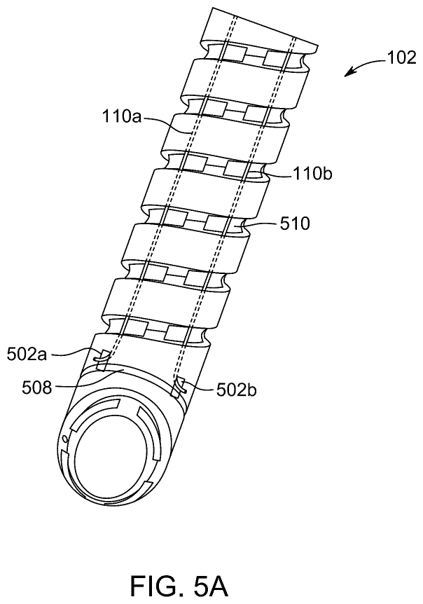

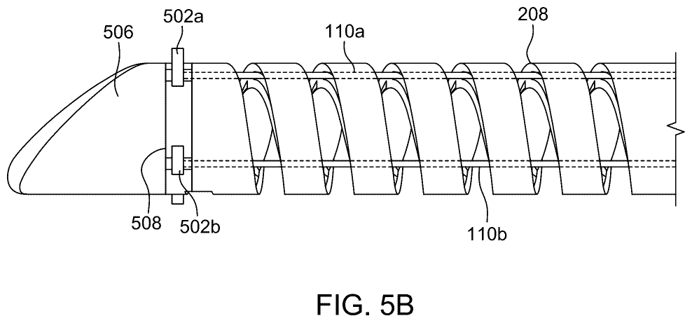

This configuration is achievable through a four navigational wires arrangement only, which is further explained in detail in conjunction with FIGS. 5A and 5B of the present invention. The navigational wires (such as 110a, 110b) are selectively activated or inactivated through the directed power from the electrical switches (120, shown in FIG. 1A) under the control of the microprocessor (118, shown in FIG. 1A).

Square wire represents a single SMA wire that is activated with an electrical current causing contraction and resulting tension. Round wire is not activated. To reduce tension from the opposite non-activated wire, the non-activated wire slides into a position that it allows it take the shortest path which is closest to the center of curvature in the plane of flexion/extension.

FIGS. 4A and 4B illustrates a schematic view of movement of the base plate 106 on the elongated endotracheal tube 102 in another embodiment of the present invention. The base plate 106 is attached around the tracheal tube airway connector 106c. The base plate 106 is moved mechanically in up and down direction as shown through FIG. 4A and FIG. 4B, respectively, which results in the movement of the navigational wires (shown in FIG. 1A). In an embodiment, a user may manually push the base plate 106 down from one side to provide tension to the opposite sides through the navigational wires (110, shown in FIG. 1A) causing bending of the bending section of the elongated endotracheal tube 102.

FIG. 5A illustrates a schematic view of an elongated endotracheal tube 100 in another embodiment of the present invention. The elongated endotracheal tube 100 includes a plurality of anchor points 502a, 502b configured on inner surface (504, shown in FIG. 2B) of the inner hollow diameter wall (206, shown in FIG. 2A).

The first wire distal end 214b is anchored at an anchor point 502a. Similarly, the second wire distal end 216b is anchored at an anchor point 502b. When the power is conducted into one of the navigational wires 110, the selective navigational wire shortens in length on application of directed power based on the navigational input unit (116, shown in FIG. 1). The other navigational wire moves via the slots to center of curvature of the elongated endotracheal tube 102 to prevent resistance to bending.

In one embodiment, the spaced apart (mechanically isolated) anchor points 502a, and 502b are connected through a conductive shunt 508 to form a conductive pathway for completing the electrical circuit. The conductive shunt 508 is configured by interconnecting a conductive layer patterned on the tubular member by metallization or etching.

Alternatively, the conductive shunt 508 is configured by forming a discrete annular insert press-fitted into the distal region of the elongated endotracheal tube. Alternatively, the conductive shunt 508 may also be configured by forming a circumferential metallic ring disposed on an outer surface (506, shown in FIG. 5B) of the tubular member.

Further, the bending section (212, shown in FIG. 2A-2C) of elongated endotracheal tube 102 is configured to have notches to eradicate the outer diameter wall (208, shown in FIG. 2A) at selected areas, and retaining the hollow inner diameter wall (206, shown in FIG. 2A) resulting in creating a helix shaped bending section, where the navigational wires 110a, 110b is directly over an outer surface 510 of the hollow inner diameter wall (206, shown in FIG. 2A).

FIG. 5B illustrates a schematic view of the elongated endotracheal tube 102 showing anchor points 502a, 502b in another embodiment of the present invention. The anchor points 502a, 502b are inserted from an outer surface 506 of the outer diameter wall 208. It will be readily apparent to those skilled in the art that various types and shapes of anchor points 502a, 502b may be envisioned without deviating from the scope of the present invention.

In an alternative embodiment, the navigational wire is metallic and in mechanical linkage with the control system (104, shown in FIG. 1A) to remove slack such as a pulley with lever. The mechanical linkage results in shortening of the navigational wire which is affixed to a flexible portion at the distal end (214b, 216b shown in FIG. 2A).

Though not shown in FIGS. 5A and 5B, the plurality of anchor points are four in number. Further, four navigational wires are configured to provide one for each anchor points. The distal end of each navigational wire is attached to separate anchor points (1-1 pairing), and the top end of each navigational wire is attached to one attachment unit. The four navigational wires results in bending the bending section in four different directions i.e. left, right, top and bottom.

The present invention offers various advantages such as allowing bending of the bending section of the endotracheal tube into the trachea. The present invention also offers a control unit to control the movement or bending of the bending section of endotracheal tube. The present invention also offers to move the bending section in four different directions.

While the above detailed description has shown, described, and pointed out novel features of the invention as applied to various embodiments, it will be understood that various omissions, substitutions, and changes in the form and details of the device or process illustrated may be made by those skilled in the art without departing from the spirit of the invention. As will be recognized, the present invention may be embodied within a form that does not provide all of the features and benefits set forth herein, as some features may be used or practiced separately from others. The scope of the invention is indicated by the appended claims rather than by the foregoing description. All changes which come within the meaning and range of equivalency of the claims are to be embraced within their scope.

1. An endotracheal tube system comprising:
an elongated endotracheal tube having an open top end, and an open distal end, the elongated endotracheal tube comprising:
a hollow inner diameter wall;
an outer diameter wall equidistantly surrounding the hollow inner diameter wall;
plurality of slots configured uniformly between the outer diameter wall and the hollow inner diameter wall, wherein the plurality of slots configured to be form across length of the hollow inner diameter wall and the outer diameter wall; and
a bending section configured near the open distal end of the elongated endotracheal tube, wherein the bending section configured to reduce amount of force required to bend the hollow inner diameter wall and the outer diameter wall;
a control system configured to attach with the elongated endotracheal tube, the control system comprising:
a base plate configured to detachably attach to the elongated endotracheal tube, the base plate having a top surface, a bottom surface, a tracheal tube airway connector configured on the top surface, and an endotracheal tube receptacle configured on the bottom surface to receive the elongated endotracheal tube;
plurality of attachment units configured to attach the base plate with the endotracheal tube; and
plurality of navigational wires comprising:
a first navigational wire comprising an elongated first wire body having a first wire top end securely attaches to a first attachment unit and a first wire distal end, the elongated first wire body runs through at least one slot across length of the elongated endotracheal tube and loops back via the slots at the bending section to securely attach with a second attachment unit at the first wire distal end;
a second navigational wire comprising an elongated second wire body having a second wire top end securely attaches to a third attachment unit and a second wire distal end, the elongated second wire body runs through at least one slot across length of the elongated endotracheal tube and loops back via the slots at the bending section to securely attach with a fourth attachment unit at the second wire distal end;
wherein the base plate is configured to pull at least one of the plurality of navigational wires to navigate the distal end of the endotracheal tube.

2. The endotracheal tube system according to claim 1, wherein the control system further comprising plurality of wire anchors, wherein each wire anchor attaches each top end of the navigational wire to the base plate.

3. The endotracheal tube system according to claim 1, wherein the control system further comprising an electrical coupling unit configured to attach with the base plate to conduct power to the navigational wires.

4. The endotracheal tube system according to claim 3 further comprising a navigational control unit configured to attach with the control system to navigate the elongated endotracheal tube.

5. The endotracheal tube system according to claim 4, wherein the navigational control unit further comprising:
a navigational input unit configured to provide navigational signals to operate the navigational wires;
a microcontroller operably coupled to the navigational input unit to process the navigational signals;
plurality of electric switches configured to direct the power to the navigational wires through the electrical coupling unit, further the plurality of electrical switches directs power based on the processed navigational signals; and
a power source to provide power to the microcontroller, the navigational input unit and the plurality of electrical switches.

6. The endotracheal tube system according to claim 5, wherein the navigational input unit comprising:
a laryngoscope housing;
a display unit configured to display video signals from a camera;
a microcontroller configured in the laryngoscope housing to process the video signals;
an electric button configured on the laryngoscope housing to provide the navigational signals to the microcontroller; and
a communication unit coupled to the microcontroller to communicate the processed navigational signals over a communication network.

7. The endotracheal tube system according to claim 3, wherein the first navigational wire shortens in length on application of directed power based on the navigational input unit, and the second navigational wire moves via the slots to center of curvature of the elongated endotracheal tube to prevent resistance to bending.

8. An endotracheal tube system comprising:
an elongated endotracheal tube having an open top end, and an open distal end, the elongated endotracheal tube comprising:
a hollow inner diameter wall;
an outer diameter wall equidistantly surrounding the hollow inner wall; and
plurality of slots configured uniformly between the outer diameter wall and the hollow inner diameter, wherein the plurality of slots configured to be form across length of the hollow inner diameter wall and the outer diameter wall;
a bending section configured near the open distal end of the elongated endotracheal tube, wherein the bending section configured to reduce amount of force required to bend the hollow inner diameter wall and the outer diameter wall;
a plurality of anchor points configured on inner surface of the hollow inner diameter wall;
a control system configured to attach with the elongated endotracheal tube, the control system comprising:
a base plate configured to detachably attach to the elongated endotracheal tube, the base plate having a top surface, a bottom surface, a tracheal tube airway connector configured on the top surface, an endotracheal tube receptacle configured on the bottom surface to receive the elongated endotracheal tube;
plurality of attachment units configured to attach the base plate with the endotracheal tube; and
plurality of navigational wires, wherein a first navigational wire comprising:
a first navigational wire comprising an elongated first wire body having a first wire top end securely attaches to a first attachment unit and a first wire distal end configured to securely attach at one of the plurality of anchors points; and
a second navigational wire comprising an elongated second wire body having a second wire top end securely attaches to a third attachment unit and a second wire distal end configured to securely attach at one of the plurality of anchors points;
wherein the base plate is configured to pull at least one navigational wire to navigate the distal end of the endotracheal tube.

9. The endotracheal tube system according to claim 8, wherein the control system further comprising an electrical coupling unit configured to attach with the base plate to conduct power to the navigational wires.

10. The endotracheal tube system according to claim 8 further comprising a navigational control unit configured to attach with the control system to navigate the elongated endotracheal tube.

11. The endotracheal tube system according to claim 8, wherein the navigational control unit further comprising:
a navigational input unit configured to provide navigational signals to operate the navigational wires;
a microcontroller operably coupled to the navigational input unit to process the navigational signals;
plurality of electric switches configured to direct the power to the navigational wires through the electrical coupling unit, further the plurality of electrical switches directs power based on the processed navigational signals; and
a power source to provide power to the microcontroller, the navigational input unit and the plurality of electrical switches.

12. The endotracheal tube system according to claim 11, wherein the navigational input unit is configured to provide wireless navigational signals.

13. The endotracheal tube system according to claim 9, wherein the first navigational wire shortens in length on application of directed power based on the navigational input unit, and the second navigational wire moves via the slots to center of curvature of the elongated endotracheal tube to prevent resistance to bending.

14. The endotracheal tube system according to claim 8, wherein the control system further comprising plurality of wire anchors, wherein each wire anchor attaches each top end of the navigational wire to the base plate.

15. The endotracheal tube system according to claim 11, wherein the navigational wires further comprising:
a third navigational wire comprising an elongated third wire body having a third wire top end securely attaches to a third attachment unit and a third wire distal end configured to securely attach at a third anchor point; and
a fourth navigational wire comprising an elongated fourth wire body having a fourth wire top end securely attaches to a fourth attachment unit and a fourth wire distal end configured to securely attach at a fourth anchor point;
wherein the navigational wires move in one of the four different directions on receiving selective conductive power under the control of microcontroller.

16. The endotracheal tube system according to claim 11, wherein the navigational input unit comprising:
a laryngoscope housing;
a display unit configured to display video signals from a camera;
a microcontroller configured in the laryngoscope housing to process the video signals;
an electric button configured on the laryngoscope housing to provide the navigational signals to the microcontroller; and
a communication unit coupled to the microcontroller to communicate the processed navigational signals over a communication network.
